(19) United States
(12) Patent Application Publication
(10) Pub. No.: US 2022/0273898 A1
(43) Pub. Date: Sep. 1, 2022

(54) ASSEMBLY FOR TRACHEAL INSTILLATION OF FLUID MEDICAMENTS IN INFANTS SUPPORTED BY NON-INVASIVE VENTILATION

(71) Applicant: Maurizio Radicioni, Perugia (IT)

(72) Inventor: Maurizio Radicioni, Perugia (IT)

(21) Appl. No.: 17/364,229

(22) Filed: Jun. 30, 2021

Related U.S. Application Data
(60) Provisional application No. 63/200,299, filed on Feb. 28, 2021.

(51) Int. Cl. A61M 16/04; A61M 25/00; A61M 25/09

(52) U.S. Cl. CPC A61M 16/0463; A61M 25/0021; A61M 25/0074; A61M 25/09; A61M 2202/0488; A61M 2205/6081; A61M 2025/09116; A61M 2240/00; A61M 2025/0042; A61M 2025/09125

(57) ABSTRACT

This assembly for tracheal instillation of fluid medicaments, particularly in preterm infants supported by non-invasive ventilation, comprises a microcatheter, a guidewire and a gripper. The microcatheter consists of a very small gauge soft composite tube with a proximal connector and a distal rounded tip. Three circumferential color-coded bands near the rounded tip and a scale in one-centimeter increments on the soft composite tube guide the insertion depth positioning of the rounded tip under direct laryngoscopy. The slidably receptive guidewire within the central lumen of the microcatheter stiffens the soft composite tube for introduction and restores softness thereto after removal. Additionally, the guidewire is locked to the microcatheter during the introduction into the trachea to avoid injury. A gripper attached to the soft composite tube improves manipulation as well as estimated insertion depth control. Once the guidewire is removed, the fluid agent (e.g. surfactant) is slowly instilled into the neonatal trachea.

### CROSS-REFERENCE TO RELATED APPLICATIONS

This application claims the benefit of U.S. Provisional patent application No. 63/200,299, filed Feb. 28, 2021, which is incorporated by reference herein in its entirety.

### FIELD OF TECHNOLOGY

The invention relates to the instillation of one or more fluid medicaments into a conduit of a person supported by non-invasive ventilation while spontaneous breathing is allowed, in order to prevent, among other things, the collapsing of his/her pulmonary alveoli. More specifically, the invention relates to the instillation of pulmonary surfactant into the trachea of the spontaneously breathing newborn or premature infant supported by continuous positive airway pressure ventilation.

### BACKGROUND OF THE INVENTION

Neonatal Respiratory Distress Syndrome (NRDS) is a common cause of respiratory distress in newborn, presenting within hours after birth, most often immediately after delivery. NRDS is caused by endogenous deficiency and/or consumption of pulmonary surfactant (PS) and primarily affects preterm neonates, with more severe disease in those smaller and more premature.

PS deficiency increases the surface tension within the small airways and alveoli, thereby reducing the compliance of the immature lung. As the surface tension increases at the alveolar level, the amount of pressure required to maintain alveolar shape increases. With reduced PS production, atelectasis occurs throughout the lung, causing reduced gas exchange. Widespread and repeated atelectasis eventually damages the respiratory epithelium, causing a cytokine-mediated inflammatory response. As pulmonary edema develops because of the inflammatory response, increasing amounts of protein-rich fluid from the vascular space leak into the alveoli, which further inactivate endogenous PS.

Many infants with NRDS require tracheal intubation with an endotracheal tube (ETT) for positive pressure invasive mechanical ventilation (IMV) and PS replacement to survive respiratory failure. While these treatment modalities have improved the outcomes for infants affected by NRDS, it continues to be a leading cause for neonatal mortality, short and long-term morbidity, as bronchopulmonary dysplasia (BPD), in preterm infants.

In the case of IMV, the ETT is used both to ventilate the newborn and to inject PS once the probe is placed in the infant's trachea. The injection is quick and easy. However, ETT mat be dangerous and cause damage, especially to the neonatal trachea, larynx or surrounding tissues. Moreover, IMV can have deleterious effects on the lung of the newborn, exacerbating the pre-existing condition and setting the stage for the development of a chronic lung disease, also known as BPD.

During the last decades perinatal management of very and extremely preterm birth improved. Consequently, most premature infants start spontaneous breathing after birth, and do not have an obligatory need for ETT and IMV. They can be stabilized with non-invasive ventilation (NIV) modalities of respiratory support, such as Continuous Positive Airway Pressure (CPAP) via nasal prongs or masks, which proved to be as efficacious as ETT with IMV and PS replacement in the management of NRDS. Today, early application of CPAP starting from the delivery room is recommended for the initial management of preterm infants with or at risk for NRDS (See e.g. Sweet D G, Camielli V, Greisen G, et al “European Consensus Guidelines on the Management of Respiratory Distress Syndrome-2019 Update” Neonatology. 2019; 115:432-450; and Committee on Fetus and Newborn; American Academy of Pediatrics “Respiratory support in preterm infants at birth” Pediatrics. 2014; 133:171-174), because it has been proven to decrease the risk of death or BPD in these patients.

However, many premature infants fail CPAP support with increased risk of mortality and major morbidities, including BPD, and still require PS replacement or IMV (See Dargaville P A, Gerber A, Johansson S, De Paoli A G, Kamlin C O, Orsini F, Davis P G; Australian and New Zealand Neonatal Network. Incidence and Outcome of CPAP Failure in Preterm Infants. Pediatrics. 2016; 138(1):e20153985. doi: 10.1542/peds.2015-3985). PS replacement has significantly improved the prognosis of premature babies since its introduction in the late 1980s. However, the increasing use of NIV modalities as first line treatment for NRDS is related to the discontinuation of PS administration in these patients and this represents a problem that needs to be addressed. To overcome this dilemma, new treatment modalities have been investigated with the aim of combining NIV with PS administration.

The Intubate-SURfactant-Extubate (INSURE) approach is a well established strategy to combine both these principles and may reduce long term respiratory adverse outcomes, as BPD, compared with CPAP alone (See e.g. Isayama T, Chai-Adisaksopha C, McDonald S D “Noninvasive Ventilation With vs Without Early Surfactant to Prevent Chronic Lung Disease in Preterm Infants: A Systematic Review and Meta-analysis” JAMA Pediatr. 2015; 169:731-739). The INSURE method consists in the administration of PS through a planned ETT and a short period of positive pressure ventilation, to favour alveolar deposition of PS, followed by prompt extubating and CPAP support. The INSURE method has been shown to be efficacious in reducing the need for IMV. However, being still related to a short period of positive pressure ventilation, it has the risk to induce lung injury. Furthermore, this approach has a high failure rate in the most vulnerable newborns, such as those with an extremely low gestational age and weight at birth (See e.g. De Bisschop B, Derriks F, Cools F “Early Predictors for Intubation-SURfactant-Extubation Failure in Preterm Infants with Neonatal Respiratory Distress Syndrome: A Systematic Review” Neonatology. 2020; 117:33-45). Therefore, there is an urgent need to research and validate further strategies capable of combining NIV modalities of respiratory support with PS replacement.

PS delivery via aerosolisation (See e.g. Rong H, Bao Y, Wen Z, Chen X, Chen C, Li F “Nebulized versus invasively delivered surfactant therapy for neonatal respiratory distress syndrome: A systematic review and meta-analysis” Medicine (Baltimore). 2020; 25; 99(48):e23113), pharyngeal instillation (See e.g. Abdel-Latif M E, Osbom D A “Pharyngeal instillation of surfactant before the first breath for prevention of morbidity and mortality in preterm infants at risk of respiratory distress syndrome” Cochrane Database Syst Rev. 2011; 16; (3):CD008311) and laryngeal mask (See e.g. Calevo M G, Veronese N, Cavallin F, Paola C, Micaglio M, Trevisanuto D “Supraglottic airway devices for surfactant treatment: systematic review and meta-analysis” J Perinatal. 2019; 39(2): 173-183) are being actively pursued in research, but have not yet been adopted to any significant degree in clinical practice.

PS administration via a thin catheter, also called as Less Invasive Surfactant Administration (LISA) method, is becoming increasingly used in neonatal intensive care units worldwide and is now recognized as a viable alternative to the standard mode of PS replacement (See Herting E, Hartel C, Gopel W. Less invasive surfactant administration: best practices and unanswered questions. Curr Opin Pediatr. 2020; 32(2):228-234. doi: 10.1097/MOP.0000000000000878). This method involves introducing a thin catheter into the neonatal trachea under direct laryngoscopy while the infant is breathing supported by CPAP with a positive end-expiratory pressure set at 6-9 cmH₂O. The distal end of the thin catheter is often introduced into the neonatal trachea via the mouth or, rarely, via the nose. PS is then slowly instilled from a syringe connected to the thin catheter within 1-2 minutes, thus avoiding the need for ETT and IMV in most treated infants. Once PS has been administered, the catheter is removed from the neonatal trachea and the infant remains supported by nasal CPAP.

The LISA method has been shown to be feasible in preterm infants down to a gestational age of 23 completed weeks (See e.g. Kribs A, Roll C, Gopel W, Wieg C, Groneck P, Laux R, Teig N, Hoehn T, Bohm W, Welzing L, Vochem M, Hoppenz M, Buhrer C, Mehler K, Stutzer H, Franklin J, Stohr A, Herting E, Roth B; NINSAPP Trial Investigators “Nonintubated Surfactant Application vs Conventional Therapy in Extremely Preterm Infants: A Randomized Clinical Trial” JAMA Pediatr. 2015; 169(8):723-730). Available data suggest that PS delivery through the LISA method decreases the risks of BPD, of death or BPD, and of CPAP failure when compared to PS administration through ETT followed by IMV and to INSURE (See e.g. Rigo V, Lefebvre C, Broux I “Surfactant instillation in spontaneously breathing preterm infants: a systematic review and meta-analysis” Eur J Pediatr. 2016; 175(12):1933-1942; and Aldana-Aguirre J C, Pinto M, Featherstone R M, et al “Less invasive surfactant administration versus intubation for surfactant delivery in preterm infants with respiratory distress syndrome: a systematic review and meta-analysis” Archives of Disease in Childhood—Fetal andNeonatal Edition 2017; 102: F17-F23). Moreover, LISA method is the sole approach where some long-term data are available (See e.g. Porath M, Karp L, Wendrich D, Dlugay V, Roth B, Kribs A “Surfactant in spontaneous breathing with nCPAP: neurodevelopmental outcome at early school age of infants ≤27 weeks” Acta Paediatr. 2011; 100(3):352-359). These data show no adverse long-term effects of the LISA method and a trend toward a better neurodevelopmental outcome.

LISA is not simply an isolated technical procedure for PS delivery but rather part of a comprehensive non-invasive approach supporting the concept of a gentle transition to the extrauterine world enabling preterm infants to benefit from the advantages of spontaneous breathing. Maintenance of spontaneous breathing with support by the LISA technique holds big promise in the care of preterm infants. Patient comfort and lower complication rates are strong arguments to further investigate and promote the LISA approach. However, many open questions remain, including the usefulness of devices/catheters up to now used to perform PS delivery through the LISA method.

Currently used devices may not ensure safe and effective PS delivery, as suggested by the animal model showing lower alveolar deposition when PS is administered via a thin catheter compared to the INSURE method (See e.g. Niemarkt H J, Kuypers E, Jellema R, Ophelders D, Hutten M, Nikiforou M, Kribs A, Kramer B W. Effects of less-invasive surfactant administration on oxygenation, pulmonary surfactant distribution, and lung compliance in spontaneously breathing preterm lambs. Pediatr Res 2014; 76(2):166-170).

The fundamental characteristics of a device that can be used to deliver PS with the LISA method have not been well defined. Ideally, this device should be easy and quick to insert into the small neonatal airway, without the need for additional aids, and made of soft material to avoid airway injuries and distortions. In addition, it should have the smallest possible caliber to ensure optimal CPAP transmission and a clearly visible surface marking, to ensure the correct position of the distal tip within the neonatal trachea under direct laryngoscopy that takes into account the variability of depth positioning according to different body weight. As far as I know, no available device to date fully meets these requirements.

Clinical experiences on the LISA method have been conducted following two main approaches: a) the so-called “Cologne Method”, which requires using small Magill's forceps to manoeuvre and introduce a thin catheter below the vocal cords (See e.g. Kribs A, Pillekamp F, Hunseler C, Vierzig A, Roth B “Early administration of surfactant in spontaneous breathing with nCPAP: feasibility and outcome in extremely premature infants (postmenstrual age ≤27 weeks)” Paediatr Anaesth. 2007; 17: 364-369); b) the so-called “Hobart Method”, which requires a 16-G catheter with 1.7 mm outer diameter (Angiocath, cat No. 382259, Becton Dickinson, Sandy, Utah, USA) which is semi-rigid and manually guidable, obviating the need for Magill's forceps (See e.g. Dargaville P A, Aiyappan A, Cornelius A, Williams C, De Paoli A G “Preliminary evaluation of a new technique of minimally invasive surfactant therapy” Arch Dis Child Fetal Neonatal Ed. 2011; 96:F243-F248). Soft catheter introduction without a forceps has been also described as the so-called “Take Care” method (See e.g. Kanmaz H G, Erdeve O, Canpolat F E, et al “Surfactant administration via thin catheter during spontaneous breathing: randomized controlled trial” Pediatrics 2013; 131: e502-e509).

Different types of catheters and instruments are used for the LISA method. According to a pan-European survey of more than 300 neonatologists from 37 European countries, standard infant feeding tubes (having side and end holes and an outer diameter of 1.7 mm) are the most commonly used devices with LISA method (56% of respondents), followed by vascular catheters (with end-hole and an outer diameter ranging from 1.3 to 1.7 mm) (34%), and suction catheters (having a central end hole and an outer diameter 1.7 mm) (15%) (See e.g. Klotz D, Porcaro U, Fleck T, Fuchs H “European perspective on less invasive surfactant administration-a survey” Eur J Pediatr. 2017; 176:147-154). The use of Foley catheters, umbilical vessel catheters, neonatal urinary catheters, and custom-made devices has also been reported. Around two-thirds of neonatologists are reported to use Magill's forceps to introduce thin catheters below the vocal cords.

There are yet no in vivo comparisons of different catheter insertion methods or catheter types for LISA method. Rigo et al. (See e.g Rigo V, Debauche C, Maton P, Broux I, Van Laere D “Rigid catheters reduced duration of less invasive surfactant therapy procedures in manikins” Acta Paediatr. 2017; 106:1091-1096) performed a simulation study in which video recordings were made of 20 neonatologists performing different methods of catheter insertion and different catheters on intubation manikins. Measures of the speed and success of insertion were derived from the recordings. The study found tracheal catheterization with a semirigid or stylet-guided catheter was successfully accomplished in a similar time to ETT and was more rapid than with a flexible tube, with and particularly without Magill's forceps. The failure rate for insertion with each of the catheters was similar (7.5-20%), which was higher than for ETT insertion (no failed insertions). When asked for their subjective evaluation, neonatologists found rigid or stylet-guided catheters the easiest to use.

To date, companies have marketed straight catheters (Lisacath®—Chiesi Pharmaceuticals) (Surfcath™— Vygon Ltd) that are easy to use orally. These catheters are stiff, so no additional devices are required to introduce the distal tip within the neonatal trachea through the vocal cords. However, stiff devices increase patient discomfort due to airways distortion during the procedure, which may trigger reflex bradycardia leading to hypoxia. Additionally, the absence of a rounded distal tip may increase the risk of airway injury with stiff devices. It should also be emphasized that the rounded distal tip must be coextruded with the distal end of the device and not glued or welded on it, due to the risk of its partial or total detachment.

Soft devices, as feeding tubes or umbilical catheters, although they protect against airway injuries during introduction into the neonatal airway, require a Magill's forceps or special introducers (Neofact®— Lyomark Pharma) (See e.g. Maiwald C A, Neuberger P, Vochem M, et al “Quick SF: a new technique in surfactant administration” Neonatology 2017; 111:211-213) to reach the tracheal lumen. This requires special technical skills and the procedure is often laborious and potentially harmful due to the increasing of patient's discomfort, which can result in acute hypoxemia and bradycardia.

Feeding tubes can lead to inadvertent loss of PS when used in the LISA method, because the PS partially adheres to the surface of the inner tube due to physical reasons and lack of tube ventilation (See e.g. De Luca D, Minucci A, Gentile L, Capoluongo E “Surfactant inadvertent loss using feeding catheters or endotracheal tubes” Am J Perinatal 2014; 31: 209-212). Moreover, these tubes as other devices have the disadvantage of side holes that are rather distant from the tip. Side holes carry the risk of increased surfactant reflux that may lead to inadvertent spillage of PS into the esophagus (See e.g. Herting E, Hartel C, Gopel W “Less invasive surfactant administration: best practices and unanswered questions” Curr Opin Pediatr. 2020; 32:228-234). Therefore, devices with a central end hole at the rounded distal tip provide more effective and waste-free PS delivery with the LISA method. Potential obstruction related to PS administration through a central end hole in thin catheters is not supported by clinical evidence. Similarly, fear of potential lung damage from expelling PS or other drug solution through the central end hole in a very thin catheter appears unwarranted if PS administration is slowly performed as prescribed in the LISA method.

Positioning of the distal tip of the device at a proper height within the neonatal trachea is crucial for effective PS delivery with the LISA technique. The correct positioning must satisfy adequate insertion of the distal tip below the vocal cords, to prevent reflux of PS into the digestive tract, as well as adequate distance from the tracheal carina, to prevent asymmetric release of PS into the bronchial tree. In general, it is recommended to insert the distal tip of the device 1-2 cm below the vocal cords (see e.g. Herting E, Hartel C, Gopel W “Less invasive surfactant administration: best practices and unanswered questions” Curr Opin Pediatr. 2020; 32:228-234). This indication may be too generic and increase the risk of inappropriate PS administration due to the shortened length of the neonatal trachea, the variability of its size at various gestational ages and birth weights, and the visualization difficulties encountered during direct laryngoscopy (e.g. narrowing of the visual field; fluid secretions; blurred vision).

The laryngoscopic visualization of the distal tip of the device passing through the laryngeal vocal chords represents the best way to be sure that the distal tip of the device has been correctly positioned at the correct height within the neonatal trachea. The small size of the device and neonatal airway, the anterior position of the neonatal larynx as the gestational age decreases, and the unfavorable conditions encountered under direct laryngoscopy often make it very difficult.

The operator often applies custom-made markers at the distal end of the device before the procedure, but this maneuver affects the sterility of the device and poses a risk to the neonate when removable markers are used. On the other hand, when available from the manufacturing process, depth markers can be confusing as suggested by a recent study (see e.g. Fabbri L, Klebermass-Schrehof K, Aguar M, Harrison C, Gulczynska E, Santoro D, Di Castri M, Line V “Five national mannequin studies found that neonatologists preferred to use LISAcath rather than Angiocath for the administration of less invasive surfactants” Acta Paediatr. 2018; 107: 780-783).

To solve these problems, several color-coded circumferential bands (at least three) could be reproduced on the distal surface of the device, reflecting the correct insertion depth of the distal tip of the device according to the different weight of the infant. The size of these bands can be borrowed from the tracheal tube markers available for different weights (from 1 kilogram to 3 kilograms), which have been widely used. These color-coded circumferential bands should be arranged on the distal surface of the device from the smallest to the largest weight starting from the central end hole of the distal tip and extending proximally. The use of complementary colors can also overcome potential visibility defects under direct laryngoscopy due to the mutual improvement of their brightness by proximity. A stained device can also improve its visualization upon introduction into the infant's airways under direct laryngoscopy. Alternatively, this improvement could also be achieved by using a colored guidewire inserted into a device with transparent walls.

It is important to highlight that the positioning of the distal tip of the device should not rely on the centimeter scale usually printed on the surface of several currently marketed devices for the LISA method. This scale can only provide an indirect estimate of the depth of insertion of the distal tip into the infant's trachea, as for tracheal intubation, according to the following rule: the depth of insertion of the tip of the device from the labial commissure must be equal to the infant's weight (expressed in kilograms) plus 6 cm. More conveniently, the centimeter scale should be used to grasp the device at an adequate distance from the distal tip to prevent it introducing too deep or too high into the infant's airway. To do this, a reference device (e.g. a gripper) attached on the catheter body may help not to lose control of the estimated insertion depth during the introduction, as well as facilitating manipulation of a thin catheter. Additionally, the centimeter scale can be used to refine distal tip positioning once the device has been introduced into the neonatal trachea.

More importantly, the thin catheter used for the LISA method represents a non-ventilable tube that can adversely affect neonatal respiratory function once introduced into the neonatal trachea by decreasing the upper airway cross-sectional area available for spontaneous breathing, preventing the transmission of CPAP pressure and increasing airway resistances. In preterm neonates (considering an inner diameter of 2-3 mm and a length of 16-20 mm) (See e.g. Jit H, Jit I “Dimensions & shape of the trachea in the neonates, children & adults in northwest India” Indian J Med Res 2000; 112: 27-33; and Fayoux P, Devisme L, Merrot O, Marciniak B “Determination of endotracheal tube size in a perinatal population: an anatomical and experimental study” Anesthesiology 2006; 104: 9546-9560; and Wani T M, Rafiq M, Akhter N, AlGhamdi F S, Tobias J D “Upper airway in infants-a computed tomography-based analysis” Paediatr Anaesth 2017; 27:501-505) the unintubated trachea has an approximate area of 3.14 mm² and airflow resistance of 40 cm H₂O/L per second. This tracheal diameter is only an estimate as it may vary depending on the measurement technique used. However, for the sake of our discussion, a tracheal diameter of less than 3 mm can be found in considerably large, although variable, proportion of preterm neonates younger than 28 weeks' gestation—i.e., those who are more at risk of negative respiratory outcomes and need careful respiratory support.

Based on their outer diameters, the thin catheters currently used for the LISA method reduce upper airway cross-sectional area available for CPAP transmission and spontaneous breathing in the very and extremely low birth weight infants from 30-40% to 70%, respectively. A further reduction of this area due to the narrowing of the neonatal airway at the cricoid ring should also be considered. Moreover, the insertion of a non-ventilable tube into the neonatal trachea increases expiratory resistance (See e.g. Matsushima Y, Jones R L, King E G, Moysa G, Alton J D M “Alterations in pulmonary mechanics and gas exchange during routine fiberoptic bronchoscopy” Chest 1984; 86:184-188), which is known to be directly proportional to the airway radius elevated to the fourth power (or the fifth power, in the case of non-laminar flow), according to the Hagen-Poiseuille law. Increased airway resistance is more prominent in infants than adults given the likelihood of crying, small trachea size and airflow turbulence due to the imperfect circular shape of the neonatal airway. Only about 30% of neonates undergoing the LISA method receive adequate analgesia or sedation (See e.g. Mehler K, Oberthuer A, Haertel C, et al “Use of analgesic and sedative drugs in VLBW infants in German NICUs from 2003-2010” Eur J Pediatr 2013; 172:1633-1639). Therefore, intense crying is more likely to occur during the procedure of PS administration via the LISA method in these infants, profoundly affecting patient's discomfort and respiratoryfunction.

Therefore, pressure transmission from CPAP support appears seriously impaired or even close to zero during PS administration with currently used devices in the LISA method, as demonstrated in a modelling study (See e.g. Jourdain G, De Tersant M, Dell'Orto V, Conti G, De Luca D “Continuous positive airway pressure delivery during less invasive surfactant administration: a physiologic study” J Perinatal 2018; 38:271-277). Conversely, it might be interesting to investigate the degree of tracheal obstruction with thinner devices that those currently marketed/patented in extremely preterm neonates with variable tracheal diameters. The inadvertent PS loss may be also reduced by using very thin catheters.

The use of a very thin catheter, hereinafter referred to as “microcatheter”, with an outer diameter s 1 mm (≤3 Fr) and made of a biocompatible soft material, stiffened for its introduction into the neonatal airways by a removable rigid guidewire sliding within its central lumen, can solve the technical problems observed in the prior art. Given the very small gauge and softness of the microcatheter, the guidewire should be positioned only within its central lumen, the same used to deliver PS, as other locations do not guarantee efficient stiffening of the device without bending and kinking during introduction into the infant's airway.

A microcatheter may be also safe and effective for tracheal instillation of other fluid medicaments in infants with NRDS, as steroids and antibiotics. Animal models suggest that tracheal instillation of a low-dose budesonide may diminish the acute lung injury of meconium aspiration syndrome (See e.g. Mokra D, Drgova A, Mokry J, et al “Combination of budesonide and aminophylline diminished acute lung injury in animal model of meconium aspiration syndrome” J Physiol Pharmacol. 2008; 59 Suppl 6:461-471). Recent meta-analysis designed to evaluate the efficacy and safety of early airway administration of corticosteroids and PS to prevent BPD in premature infants with NRDS reported that early tracheal instillation of corticosteroids using PS as the vehicle is an effective and safe option for preventing BPD, decreasing the additional PS usage and reducing mortality (See e.g. Zhong Y Y, Li J C, Liu Y L, et al “Early Intratracheal Administration of Corticosteroid and Pulmonary Surfactant for Preventing Bronchopulmonary Dysplasia in Preterm Infants with Neonatal Respiratory Distress Syndrome: A Meta-analysis” Curr Med Sci 2019; 39:493-499 and Venkataraman R, Kamaluddeen M, Hasan S U, et al “Intratracheal Administration of Budesonide-Surfactant in Prevention of Bronchopulmonary Dysplasia in Very Low Birth Weight Infants: A Systematic Review and Meta-Analysis” Pediatr Pulmonol. 2017; 52:968-975). Acute lung injury, such as meconium aspiration syndrome in neonates, may present with exacerbated ventilation and perfusion abnormalities. This can impair the efficacy of intravenous antibiotic therapy in treating pulmonary infection. Tracheal instillation via perfluorochemical agents as a vehicle through microcatheters may adequately deliver drugs, such as the poorly pulmonary-penetrative antibiotic vancomycin, to affect lung regions while maintaining gas exchange and non-toxic serum level. A microcatheter may be also used for tracheal instillation of prostacyclin to improve oxygenation without systemic vascular repercussions in infants with persistent pulmonary hypertension supported by NIV (See e.g. De Jaegere A P, van den Anker J N “Endotracheal instillation of prostacyclin in preterm infants with persistent pulmonary hypertension” Eur Respir J. 1998; 12(4):932-934).

### OBJECT OF THE INVENTION

The object of the present invention is to overcome technical issues concerning the device to use in the LISA method as emerged from the review of the prior art. The innovation focuses on solving not fully addressed problems, as to ensure adequate CPAP pressure transmission and lower respiratory workload in patients, particularly in the very and extremely low birth weight infants, to prevent potential anatomical distortion and airway injuries, and to facilitate introduction and ensure the correct positioning of the distal tip into the trachea under direct laryngoscopy.

The novelty of the present invention is represented by an assembly comprising a microcatheter consisting of a soft composite tube stiffened by a removable guidewire sliding inside its central lumen, and a gripper that can be attached to the soft composite tube of the microcatheter. The peculiarities of components and materials and their use in this assembly improve safety, efficacy and tolerability of PS administration with the LISA method, particularly in the very and extremely low birth weight infants.

### SUMMARY OF THE INVENTION

An object of the invention is to provide a suitable device that allows one or more fluid medicaments to be instilled easily and quickly into a conduit of a person supported by NIV modalities while allowing spontaneous breathing. More particularly, the assembly described here aims to provide easy and quick introduction into the infant's trachea and effective endotracheal instillation of PS via the LISA method in the autonomous breathing infant with NRDS supported by CPAP improving CPAP pressure transmission and avoiding airway injury and distortions.

Representatively, the assembly comprises a microcatheter, a guidewire and gripper. Schematically, the function of this assembly is to ensure quick, safe and correct positioning of the distal end of the microcatheter within the tracheal lumen for effective PS administration without hindering CPAP transmission and spontaneous breathing, thus increasing the patient's tolerability for the procedure, particularly in very and extremely low birth weight infant.

The microcatheter of the assembly consists of a soft composite tube with a very small gauge, having a proximal and a distal end and a central lumen open at the proximal and distal ends, the proximal end being provided with a proximal connector in fluid communication with the central lumen and configured to allow connection of a threaded plastic cap and of a means for PS instillation, and the distal end terminating in a rounded tip having a central end hole, the rounded tip being coextruded with the distal end and the central end hole being in fluid communication with the central lumen.

The guidewire of the assembly is “stiff-flexible” and slidably receptive into the central lumen of the soft composite tube of the microcatheter to provide rigidity thereto for introduction into the infant's airway and restore softness thereto after removal. The guidewire has a proximal, a distal ends, and a length relative to the microcatheter, the proximal end being welded to a threaded plastic cap and the distal end being rounded. The threaded plastic cap can be screwed around the proximal connector of the microcatheter to lock the guidewire during the introduction of the assembly into the infant's airway to avoid trauma. As used herein “stiff-flexible” refers to the property of the guidewire resulting from its small gauge and material allowing the guidewire to be bent or flexed, with the guidewire providing some resistance to the change in shape.

The gripper of the assembly has a longitudinal medial groove for the attachment to any point of the soft composite tube of the microcatheter, to facilitate manipulation as well as acting as a reference device on the microcatheter to maintain control of the estimated insertion depth of the rounded tip within the tracheal lumen during the introduction under direct laryngoscopy.

Some preferred but non-limiting features of the assembly are as follows:

the proximal connector is of clear rigid plastic,

the soft composite tube of the microcatheter has a uniform outer diameter preferably of 0.8 mm (2.4 Fr) and in any case not exceeding 1 mm (≤3 Fr) and is between 15 cm and 25 cm long,

the soft composite tube of the microcatheter is provided with a sequence of circumferential color-coded bands on the surface near the rounded tip (preferably three) that are sized according to different infant's weight (from s 1 kilogram to ≥3 kilograms) and arranged from the smallest to the largest weight starting from the central end hole of the rounded tip and extending proximally along the soft composite tube providing the visual assessment of the depth positioning of the rounded tip of the microcatheter into the neonatal trachea during the introduction, and a scale in one-centimeter increments on the surface length providing an additional assessment/refinement of the depth positioning of the rounded tip,

the soft composite tube of the microcatheter is preferably of clear material as polyurethane, or polyvinyl chloride, or high-density polyethylene or flexible polyamide without plasticizer; alternatively, the wall of the soft composite tube of the microcatheter can also be colored, preferably blue,

the guidewire has a uniform outer diameter preferably of 0.53 mm (1.6 Fr) and in any case not exceeding 0.7 mm (≤2.1 Fr),

the guidewire is colored, preferably blue, in the case of soft composite tube with clear wall, and made of metal, preferably tungsten or nitinol,

the gripper is preferably of semi-hard rubber.

To perform PS administration via the LISA method, the assembly is introduced into the infant's airway under direct laryngoscopy guidance. Once the color-coded circumferential band, chosen based on the infant's weight, passes between the laryngeal vocal cords and disappears under the glottic plane, the rounded tip of the soft composite tube of the microcatheter is placed at the optimum height within the tracheal lumen for effective PS administration and any further advancement of the assembly is stopped. The laryngoscope is then removed from the infant's mouth, and the guidewire unlocked and removed from the microcatheter. Thereafter, PS is slowly instilled into the trachea within 1-2 minutes by connecting the cone of a syringe filled with the prescribed amount of PS to the proximal connector of the microcatheter. At the end of the procedure, the microcatheter is removed from the infant's airway. All of the above steps are performed with the infant breathing autonomously without interrupting CPAP support.

The very small gauge soft composite tube of the microcatheter ensures the optimal airway patency for CPAP transmission with minimal increase in airway resistance, which lower patient's respiratory workload during the LISA procedure particularly in very and extremely low birth weight infant. Stiffening the soft composite tube of the microcatheter with an internal guidewire facilitates rapid insertion of the rounded tip of the microcatheter into the infant's airway without the need for additional means, even in very and extremely low birth weight infant. The gripper attached to the soft composite tube of the microcatheter facilitates manipulation of the assembly as well as providing a reference device to maintain control of the estimated insertion depth during the introduction of assembly under direct laryngoscopy, and an additional refinement of the depth positioning of the rounded tip can be performed using the scale in one-centimeter increments at the end of the introduction of the assembly. The sequence of three color-coded circumferential bands near the rounded tip of the soft composite tube and the staining of the soft composite tube or the colored guidewire (in the case of a soft composite tube with clear walls) improve visualization under direct laryngoscopy ensuring correct placement of the rounded tip of the microcatheter within the tracheal lumen. The rounded tip coextruded with the distal end of the soft composite tube prevents airway injury during positioning of the assembly and eliminates the risk of detachment into the infant's trachea. Locking the guidewire to the proximal connector of the microcatheter and the rounded distal end of the guidewire prevent airway injury and wall ruptures of the soft composite tube during the introduction of the assembly, respectively. Moreover, the removal of the guidewire from the central lumen of the soft composite tube allows the same to adapt to the infant's airway during PS delivery avoiding significant anatomical distortions during the instillation of PS, thus increasing patient's tolerability for the procedure.

### BRIEF DESCRIPTION OF THE SEVERAL VIEWS OF THE DRAWINGS

The foregoing aspects and other features of the invention are explained in the following description, taken in connection with the accompanying drawings, wherein:

FIG. 1 is an elevation view illustrating the features of the assembly of the present invention prepared for introduction into the infant's trachea. The gripper is shown attached to the soft composite tube of the microcatheter at a distance between its distal edge and the rounded tip corresponding to 6 centimeters plus the weight (expressed in kilograms) of the infant who will receive PS via the LISA method. The guidewire is shown within the central lumen of the microcatheter to stiffen the soft composite tube for introduction of the assembly into the infant's trachea and is visible throughout the length of the clear wall of the microcatheter. The threaded plastic cap welded to the proximal end of the guidewire is screwed around the proximal connector of the microcatheter to secure the guidewire for safe introduction. Also shown are three circumferential color-coded bands on the surface of the soft composite tube near the rounded tip, the scale in one-centimeter increments on the surface of the soft composite tube, the outer diameter of the soft composite tube and the inner diameter of the central lumen/end hole at the rounded tip;

FIG. 2 FIG. 2 is an elevation view, similar to FIG. 1, illustrating the guidewire partially withdrawn from the central lumen of the microcatheter once the threaded plastic cap has been unscrewed from the proximal connector of the microcatheter by unlocking the guidewire therefrom;

FIG. 3 is a perspective view of the distal portion of the soft composite tube of the microcatheter with the guidewire inserted within its central lumen, illustrating the sequence of three color-coded circumferential bands used for positioning the rounded tip of the microcatheter within the tracheal lumen. These bands are located near the rounded tip having a central end hole, sized according to three different weights (from s 1 kilogram to z 3 kilograms) and arranged from the smallest to the largest weight starting from the central end hole of the rounded tip and extending proximally along the soft composite tube;

FIG. 4 is an elevation view of the rear of the gripper illustrating the longitudinal medial groove for the attachment to any point of the soft composite tube of the microcatheter;

FIG. 5 is an elevation view of the rear part of the gripper showing a section of the soft composite tube of the microcatheter, internally stiffened by the guidewire, housed within the longitudinal medial groove of the gripper;

FIG. 6 is a cross-sectional view of the upper airway and esophagus of an autonomously breathing infant with NRDS supported by CPAP via a nasal interface applied to the nostrils. The optimal height within the tracheal lumen for positioning of the rounded tip of the soft composite tube of the microcatheter for effective PS delivery, avoiding asymmetric distribution into the bronchial tree and esophageal spillage is shown;

FIG. 7 is a cross-sectional view of the same infant of FIG. 6, after a laryngoscope has been placed into his/her mouth, illustrating the rounded tip of the soft composite tube of the microcatheter, as shown in FIG. 1, reaching the correct position within the tracheal lumen for effective PS administration;

FIG. 8 is a cross-sectional view of the same infant of FIG. 7, after the laryngoscope has been removed from his/her mouth, illustrating the soft composite tube of the microcatheter, as shown in FIG. 2, adapting to the infant's airway as the guidewire is withdrawn from the microcatheter;

FIG. 9 is a cross-sectional view of the same infant of FIG. 7 after removal of the guidewire from the microcatheter, as shown in FIG. 8, has been fully carried out, illustrating a syringe filled with the prescribed amount of PS connected to the microcatheter for slow tracheal instillation through the central lumen according to the LISA method.

### DETAILED DESCRIPTION OF THE INVENTION

The invention provides, among other things, an assembly for easily and quickly instilling one or more fluid medicaments into a conduit of a person supported by NIV modalities, in particular into the trachea of a newborn or preterm infant supported by nasal CPAP while spontaneous breathing is allowed. In the following, the invention will be more particularly described in the case of the tracheal instillation of PS preferably in very and extremely low birth weight infant. However, this is not limiting as the assembly can be used for instilling other types of fluid medicaments and/or be used for adults.

In this section, we shall explain this invention with reference to the appended drawings. Moreover, in the following description, “proximal” will denote a part that is located near the operator when the latter uses the assembly of the invention, while “distal” will denote a part that is away from the operator during this use.

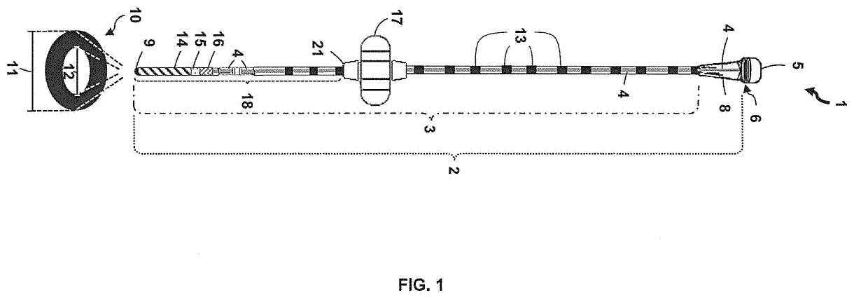

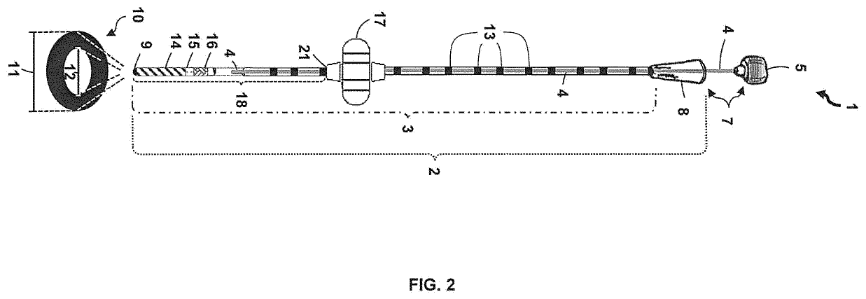

Referring herein mainly to FIG. 1-2 until otherwise indicated, the assembly 1 of this disclosure comprises a microcatheter 2, a guidewire 4 and a gripper 17.

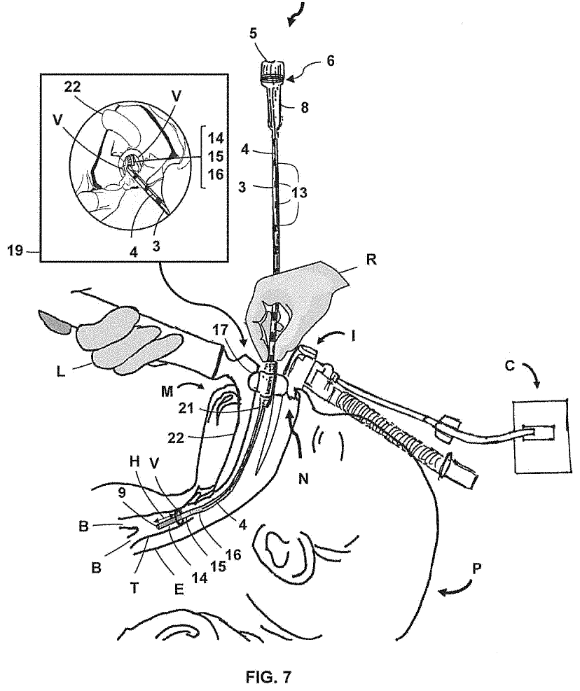

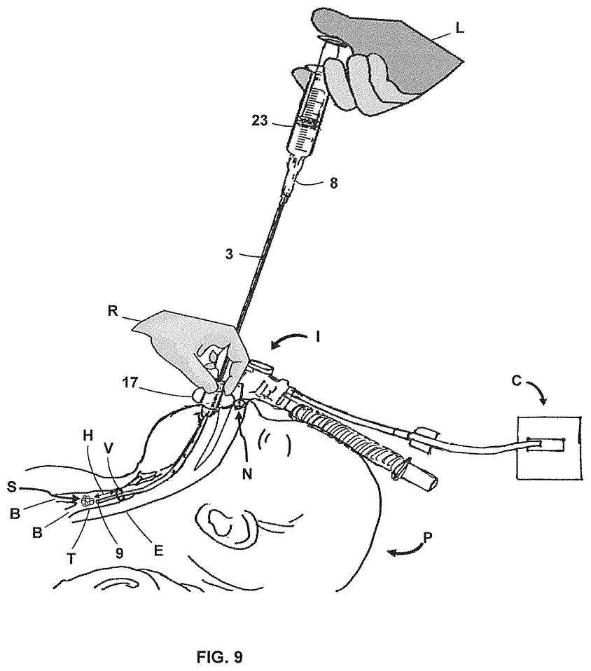

The microcatheter 2 consists of a soft composite tube 3 of straight, uniform and very small gauge (outer diameter 11≤1 mm), with transparent walls, having a proximal and a distal end and a central lumen open at the proximal and distal ends. The proximal end of the soft composite tube 3 is provided with a proximal connector 8 in fluid communication with the central lumen of the soft composite tube 3. The proximal connector 8 is configured to cooperate with the tip of a syringe comprising PS, as shown in FIG. 9, and to allow connection of a threaded plastic cap 5, as shown in FIG. 1 and FIG. 7. The proximal connector 8 can be of the Luer, Luer Lock type or any connector conforming to the standards in force. Suitable material for the proximal connector 8 is preferably clear rigid plastic. The distal end of the soft composite tube 3 ends in a rounded tip 9 having a central end hole 11, which has an inner diameter 12 not exceeding 0.7 mm. The rounded tip 9 is coextruded with the distal end of the soft composite tube 3 and the central end hole 10 is in fluid communication with the distal end of the central lumen of the soft composite tube 3. Suitable biocompatible materials for the soft composite tube 3 include polyurethane, polyvinyl chloride, high-density polyethylene and flexible polyamide without plasticizer.

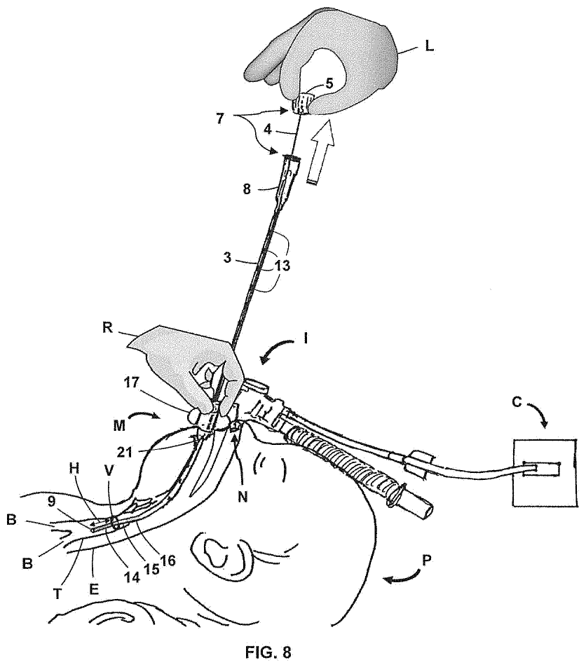

Due to its very small gauge, the soft composite tube 3 of the microcatheter 2 ensures the optimal airway patency for CPAP C transmission with minimal increase in airway resistance, thus lowering the respiratory workload during the LISA procedure, particularly in very and extremely low birth weight infant, as shown in FIG. 7-9. The coextruded rounded tip 9 of the soft composite tube 3 of the microcatheter 2 prevents airway injury and eliminates the risk of its detachment into the trachea during the introduction of the assembly 1, respectively. Moreover, once the guidewire 4 is removed from the microcatheter 2, the soft composite tube 3 adapts to the infant's airway avoiding significant anatomical distortions during PS instillation, thus increasing tolerability for the procedure, as shown in FIG. 8 and FIG. 9.

The guidewire 4 has a straight, uniform and very small gauge with an outer diameter ≤0.7 mm, a proximal and a distal end and a length relative to the microcatheter 2. The guidewire 4 is stiff-flexible and slidably receptive into the central lumen of the microcatheter 2. The proximal end of the guidewire 4 is welded to a threaded plastic cap 5 and the distal end has a rounded shape. The threaded plastic cap can be screwed around the proximal connector 8 of the microcatheter 2, as shown in FIG. 1 and in FIG. 7, or unscrewed therefrom, as shown in FIG. 2 and in FIG. 8. By screwing the threaded plastic cap 5 around the proximal connector 8 of the microcatheter 2 the guidewire 4 is locked 6 for safe introduction. The guidewire 4 is preferably of blue color to improve visualization of the assembly 1 under direct laryngoscopy 19, as shown in FIG. 7. The most suitable material for guidewire 4 is metal, especially tungsten or nitinol.

The guidewire 4 stiffens the soft composite tube 3 of the microcatheter 2 for easy and quick introduction of the assembly 1 into the infant's trachea without the need for additional aids, even in very and extremely low birth weight infant, avoiding significantly kinking and/or bending. Due to its small gauge, the guidewire 4 is also enough flexible to avoid airway injury during the introduction. The operator can also shape the assembly 1 by bending it distally in the most difficult cases, exploiting the rigidity of the guidewire 4. The locking 6 of the guidewire 4 to the proximal connector 8 of the microcatheter 2 prevents the distal end from coming out of the central end hole 10 of the rounded tip 9 during the introduction of the assembly 1 into the infant's airway under direct laryngoscopy 19, as shown in FIG. 7, further avoiding potential airway injury. Moreover, the rounded shape of the distal end of the guidewire 4 prevents potential ruptures of the thin-walled soft composite tube 3.

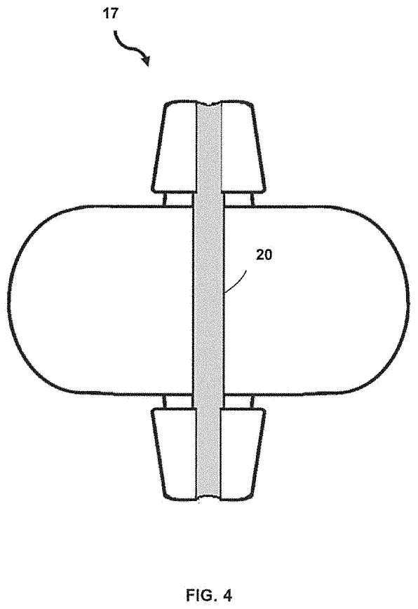

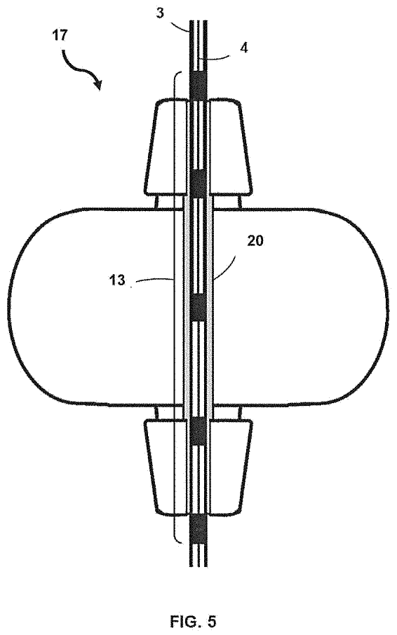

FIG. 4 shows the rear part of the gripper 17, preferably of semi-hard rubber, with the longitudinal medial groove 20 suitable for hooking a portion of the soft composite tube 3 when internally stiffened by the guidewire 4. The gripper 17 can be attached in a reversible way to any point of the soft composite tube 3 of the microcatheter 2 by pressing a portion of the soft composite tube 3 against the longitudinal medial groove 20 until completely seated inside, as more clearly shown in FIG. 5. In fixing the gripper 17 on the soft composite tube 3, care should be taken in placing the distal edge 21 of the gripper 17 at a distance 18 from the rounded tip 9 equal to 6 cm plus the infant's weight (expressed in kilograms), using the scale 13 in one-centimeter increments of the soft composite tube 3 as a reference, as shown in FIG. 1-2. If this is adhered to, it will prevent the rounded tip 9 of the soft composite tube 3 of the microcatheter 2 from being inserted too distally into the infant's airway, because the gripper 17 stops any further advancement of the assembly 1 once reached the labial commissure of the mouth M of the infant P, as shown in FIG. 7-9. Similarly, a deficit of insertion should always be considered and ruled out when the distal edge 21 of the gripper 17 is positioned too far from the infant's mouth after the introduction of the microcatheter 2 into the infant's trachea. If necessary, adjustments of the insertion depth of the rounded tip 9 can be performed using the scale 13 in one-centimeter increments on the surface of the soft composite tube 3 as a reference.

The gripper 17 provides also a valid reference device on the soft composite tube 3 of the microcatheter 2 that helps the operator not to lose the control of the estimated insertion depth 18 of the rounded tip 9 while introducing the assembly 1 into the infant's airway under direct laryngoscopy 19, as well as facilitating the manipulation of the microcatheter 2.

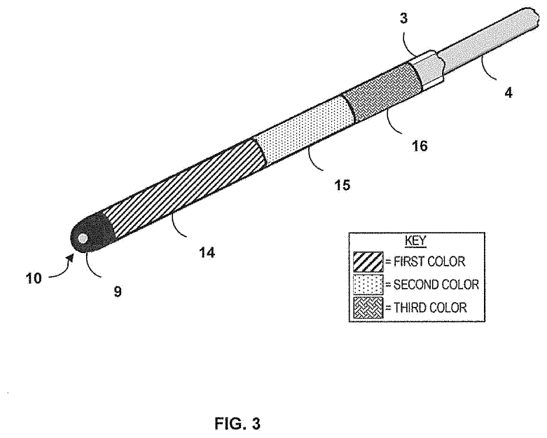

FIG. 3 illustrates, with more detail respect to FIG. 1-2, 7-9, the sequence of the circumferential color-coded bands (preferably three) 14 15 16 on the distal portion of the soft composite tube 3 for determining the insertion depth of the rounded tip 9 under direct laryngoscopy. These bands 14 15 16 are sized according to three different weights (from ≤1 kilogram to ≥3 kilograms) and arranged from the smallest to the largest weight starting from the central end hole 10 of the rounded tip 9 and extending proximally on the soft composite tube 3.

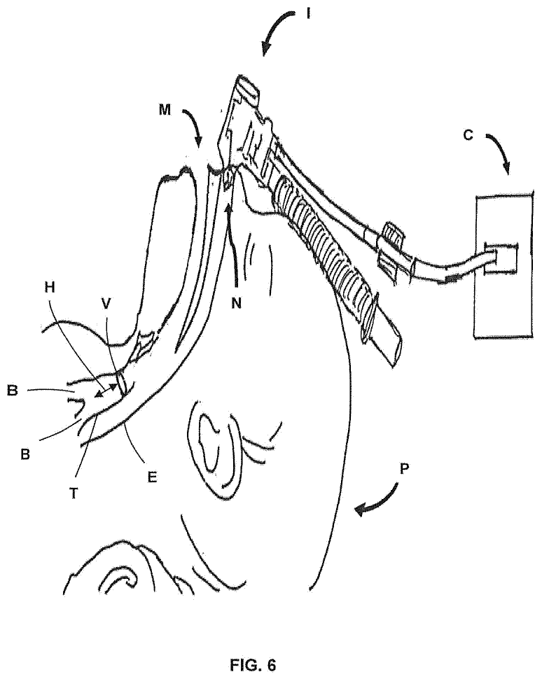

The color choice and arrangement of the three circumferential color-coded bands 14 15 16 depend on their optimal visibility under direct laryngoscopy 19, as shown in FIG. 7, due to the mutual improvement of brightness of the complementary colors by proximity. The circumferential color-coded bands 14 15 16 aim to ensure the correct placement of the rounded tip 9 at the optimal height H within the tracheal lumen T, which prevents asymmetrical administration of PS into the tracheobronchial tree B (due to a deep positioning) and esophageal spillage E (due to a high positioning), as shown in FIG. 6-9. The sizes of the three circumferential color-coded bands 14 15 16 shown in FIG. 1-3, 7-9 are borrowed from the distal markers of the commonly used neonatal tracheal tubes that are available for different weights (from s 1 kilogram to Z 3 kilograms). Proceeding proximally from the rounded tip 9 on the soft composite tube 3 of the microcatheter 2, the arrangement of the circumferential color-coded bands 14 15 16 follows this order:

the first circumferential color-coded band 14, preferably of bright purple, extends 1.8 cm proximally along the surface of the composite tube soft 3 from the central end hole 10 of the rounded tip 9, representing the optimal insertion depth H inside the tracheal lumen T below the laryngeal vocal cords V for an infant weighing 1,000 grams or less, as shown in FIG. 6-9;

the second circumferential color-coded band 15, preferably of bright yellow, extends 0.5 cm proximally along the surface of the composite tube soft 3 from the proximal edge of the first circumferential color-coded band 14, representing the optimal insertion depth H inside the tracheal lumen T below the laryngeal vocal cords V for an infant weighing between 1,000 to 2,000 grams, as shown in FIG. 6-9;

the third circumferential color-coded band 16, preferably of bright red, extends 0.5 cm proximally along the surface of the composite tube soft 3 from the proximal edge of the second circumferential color-coded band 15, representing the optimal insertion depth H inside of the tracheal lumen T below the laryngeal vocal cords V for an infant weighing 3,000 grams or more, as shown in FIG. 6-9.

Herein it is briefly described the LISA method performed with the assembly 1 of this disclosure.

FIG. 6 illustrates a spontaneously breathing infant P of 1 kilogram supported by CPAP C through an interface I applied to the nostrils N who will receive PS via the LISA method to treat NRDS. Anatomical landmarks as vocal cords V, main bronchial tree B, esophagus E and optimal height H within the tracheal lumen T where the distal tip of the device used should be positioned for effective PS delivery are shown.

The assembly 1 of this disclosure is prepared for introduction into the infant's airway by inserting the guidewire 4 into the proximal connector 8 and sliding it into the central lumen of the microcatheter 2 until the threaded plastic cap 5 meets the proximal connector 8. At this point, the operator screws it 5 around proximal connector 8 to lock 6 the guidewire 4 for safe introduction of the assembly 1, as shown in FIG. 1. Thereafter, the operator attaches the gripper 17 to the microcatheter 2 by pressing a portion of the soft composite tube 3 stiffened by the guidewire 4 against the longitudinal medial groove 20 on its rear until completely seated inside, as shown in FIG. 5. In doing this, the operator pays particular attention in placing the distal edge 21 of the gripper 17 at a distance 18 equal to 7 cm from the rounded tip 9 using the scale 13 in one-centimeter increments on the surface of the soft composite tube 3 as a reference, as shown in FIG. 1-2. This, among other things, will help the operator maintain control of the estimated insertion depth 18 while introducing the assembly 1 into the infant's trachea under direct laryngoscopy 19.

Referring now to FIG. 7, the operator introduces the laryngoscope blade 22 using the left-hand L into the mouth M of the same infant P of FIG. 6 to visualize the laryngeal vocal cords V under direct laryngoscopy 19 without interrupting CPAP C support applied to the nostrils N via the interface I. By grasping the assembly 1 at the gripper 17 with the right-hand R, the operator slides the rounded tip 9 of the soft composite tube 3 of the microcatheter 2 between the vocal cords V of the newborn and enters the tracheal lumen T. Once the proximal edge of the first circumferential color-coded band 14 disappears under the vocal cords V, the operator stops any further advancement of the assembly 1, as the rounded tip 9 is now positioned at the optimum height H within the tracheal lumen T that avoids both asymmetric administration into the bronchial tree B and esophageal leakage E of PS.

Referring now to FIG. 8, the operator takes off the laryngoscope blade 22 from the mouth M of the same infant P of FIG. 7 using the left-hand L, while firmly holding the gripper 17 at the labial commissure of the infant's mouth M with the right-hand R. At this point, the operator checks the position of the distal edge 21 of the gripper 17 relative to the labial commissure of the mouth M of the infant P. If necessary, a further refinement of the insertion depth of the rounded tip 9 can be now performed, based on the estimated insertion depth 18, as shown in FIG. 1-2, using the scale 13 in one-centimeter increments on the surface of the soft composite tube 3 as a reference. Once confirmed that the rounded tip 9 has been placed at the optimum height H within the tracheal lumen T to avoid either asymmetric administration B or esophageal leakage E of PS, the operator unscrews the threaded plastic cap 5 from the proximal connector 8 to unlock 7 the guidewire 4. Thereafter, the operator removes the guidewire 4 from the microcatheter 2 using the left-hand L, while still holding firmly the gripper 17 to the labial corner of the mouth M of the newborn P with the right-hand R, always without interrupting the CPAP C support applied to the nostrils N through the interface I.

Referring now to FIG. 9, the tip of a syringe 23 filled with the prescribed amount of PS heated to body temperature plus an additional 1 ml of air is connected to the proximal connector 8 of the microcatheter 2. PS is then slowly delivered S at the rounded tip 9 of the soft composite tube 3 of the microcatheter 2 within the tracheal lumen T in 1-2 minutes, while the spontaneously breathing infant P remains supported by CPAP C applied to the nostrils N through the interface I. At the end of the procedure, the microcatheter 2 is removed and the infant P remains supported by CPAP C, as shown in FIG. 6.

In the preceding detailed description, specific embodiments are described. As various modifications and changes could be made thereto without departing from the broader spirit and scope of the invention, it is intended that all matter contained in the description above or shown in the accompanying drawings should be interpreted as illustrative and not in a limiting sense.

What I claim is:

1. An assembly (1) for the instillation of one or more fluid medicaments into the trachea (T) of a person while spontaneous breathing is allowed, particularly the person being a newborn or premature infant M supported by CPAP (C) and receiving pulmonary surfactant (S), the assembly (1) comprising:
a microcatheter (2), the microcatheter (2) consisting of a rigid connector (8) and a soft composite tube (3) with proximal and distal ends and a central lumen open at the proximal and distal ends, the soft composite tube (3) being characterized by straight, uniform outer diameter (11) not exceeding 1 mm (≤3 Fr), allowing for optimal CPAP (C) transmission in the newborn (P), the distal end terminating in a coextruded rounded tip (9), avoiding airway injury and detachment of foreign bodies during introduction of the microcatheter (2) into the trachea (T), the coextruded rounded tip (9) bearing a central end-hole (10), the central end-hole (10) being in fluid communication with the central lumen, a sequence of color-coded circumferential bands (14-16) near the coextruded rounded tip (9), ensuring positioning of the coextruded rounded tip (9) at the optimum height (H) inside the trachea (T) under direct laryngoscopy (19), sized and arranged according to different body weights of the newborn (P), and a scale in one-centimeter increments (13) along the surface;
a guidewire (4), the guidewire (4) characterized by being stiff-flexible and slidably receptive into the microcatheter (2), to stiffen the soft composite tube (3) during introduction of the microcatheter (2) into the trachea (T) and restore softness thereto after removal, having proximal and distal ends and length relative to the microcatheter (2), the proximal end being welded to a threaded plastic cap (5), and the distal end being rounded; and
a gripper (17), the gripper (17) being characterized by the reversible attachment to any point of the soft composite tube (3) of the microcatheter (2), when stiffened internally by the guidewire (4), through a longitudinal medial groove (20) on its rear, the longitudinal medial groove (20) being sized to engage within it a portion of the soft composite tube (3).

2. The assembly (1) as in claim 1, wherein the connector (8) being hollow, made of clear rigid material and with proximal and distal ends, the proximal end being configured to allow connection of the threaded plastic cap (5) and of a means (23) for instillation of pulmonary surfactant (S) and other fluid medicaments, the distal end being welded to the proximal end of the soft composite tube (3) and in fluid communication with the central lumen of the soft composite tube (3).

3. The assembly (1) as in claim 1, wherein the sequence of color-coded circumferential bands (14-16) on the soft composite tube (3) comprising:
a first color-coded circumferential band (14), the first color-coded circumferential band (14) ensuring the position of the coextruded rounded tip (9) at the optimum height (H) within the trachea (T) for newborn (P) weighing ≤1 kg, characterized by being preferably of bright purple and extending 1.8 cm proximally along the surface of the composite tube soft (3) from the central end hole (10) of the coextruded rounded tip (9);
a second color-coded circumferential band (15), the second color-coded circumferential band (15) ensuring the position of the coextruded rounded tip (9) at the optimum height (H) within the trachea (T) for newborn (P) weighing between 1 kg and 2 kg, characterized by being preferably of bright yellow and extending 0.5 cm proximally along the surface of the composite tube soft (3) from the proximal edge of the first color-coded circumferential band (14); and
a third color-coded circumferential band (16), the third color-coded circumferential band (16) ensuring the position of the coextruded rounded tip (9) at the optimum height (H) within the trachea (T) for newborn (P) weighing ≥3 kg, characterized by being preferably of bright red and extending 0.5 cm proximally along the surface of the composite tube soft (3) from the proximal edge of the second color-coded circumferential band (15).

4. (canceled)

5. (canceled)

6. The assembly (1) as in claim 1, wherein the soft composite tube (3) having the outer diameter (11) preferably 0.8 mm (2.4 Fr), to allow optimal CPAP (C) transmission in the very and extremely low birth weight newborn (P), and wherein the sequence of color-coded circumferential bands (14-16) being colored with complementary colors.

7. The assembly (1) as in claim 1, wherein the walls of the soft composite tube (3) of the microcateter (2) are clear or blue colored, to improve visualization during the introduction of the microcatheter (2) into the trachea (T) under direct laryngoscopy (19).

8. The assembly (1) as in claim 1, wherein the guidewire (4) having straight, uniform outer diameter not exceeding 0.7 mm (≤2.1 Fr), preferably 0.53 mm (1.6 Fr), and wherein the locking (6) of the guidewire (4) to the connector (8), obtained by screwing the threaded plastic cap (5) around the proximal end of the connector (8), and the rounded distal end of the guidewire (4) preventing airway injury and ruptures of the soft composite tube (3) during the introduction of the microcatheter (2) into the trachea (T).

9. The assembly (1) as in claim 1, wherein the guidewire (4) being made of rigid material, preferably metal such as tungsten or nitinol, to facilitate the introduction of the microcatheter (2) into the trachea (T), and wherein the removal of the guidewire (4) avoiding anatomical distortion of the trachea (T) during the instillation of pulmonary surfactant (S) and other fluid medicaments into the trachea (T) by restoring softness to the soft composite tube (3), and further wherein being colored, preferably of blue, if the walls of the soft composite tube (3) are clear, to improve visualization during the introduction of the microcatheter (2) into the trachea (T) under direct laryngoscopy (19).

10. The assembly (1) as in claim 1, wherein the guidewire (4) being coated with a thin biocompatible silicone film, preferably during manufacturing, to prevent the walls of the soft composite tube (3) from kinking during its removal from the microcatheter (2).

11. The assembly (1) as in claim 1, wherein the attachment of the gripper (17) on the soft composite tube (3) facilitates handling of the microcatheter (2) during the introduction the microcatheter (2) into the trachea (T) under direct laryngoscopy (19).

12. The assembly (1) as in claim 1, wherein the attachment of the gripper (17) on the soft composite tube (3), using the scale in one-centimeter increments (13) as a reference, allowing the operator to maintain constant control of the estimated insertion depth (18) during the introduction of the microcatheter (2) into the trachea (T) under direct laryngoscopy (19).

13. The assembly (1) as in claim 1, wherein the gripper (17) being preferably made of semi-hard rubber.

14. The assembly (1) as in claim 1, wherein the soft composite tube (3) being made of biocompatible material, preferably polyurethane, or polyvinyl chloride, or high-density polyethylene or flexible polyamide without plasticizer, and wherein being between 15 and 25 cm in length.
